(19) United States
(12) Patent Application Publication
(10) Pub. No.: US 2018/0170841 A1
(43) Pub. Date: Jun. 21, 2018

(54) PROCESSES AND APPARATUSES FOR TOLUENE METHYLATION IN AN AROMATICS COMPLEX

(71) Applicant: UOP LLC, Des Plaines, IL (US)

(72) Inventors: Robert J. Schmidt, Barrington, IL (US); Feng Xu, Arlington Heights, IL (US); Joseph A. Montalbano, Elmhurst, IL (US); Ling Zhou, Palatine, IL (US); Edwin P. Boldingh, Arlington Heights, IL (US); Linda S. Cheng, Highland Park, IL (US); John J. Senetar, Naperville, IL (US)

(21) Appl. No.: 15/848,220

(22) Filed: Dec. 20, 2017

Related U.S. Application Data
(60) Provisional application No. 62/437,006, filed on Dec. 20, 2016.

(51) Int. Cl. C07C 15/08; C07C 1/24; C07C 41/09; C07C 31/04; C07C 43/04; B01J 8/00; B01D 3/14

(52) U.S. Cl. CPC C07C 15/08; C07C 1/24; C07C 41/09; C07C 31/04; C07C 43/043; C07C 15/06; B01D 3/143; C10G 2300/1088; C10G 2300/1096; C10G 2400/30; B01J 8/0005

(57) ABSTRACT

This present disclosure relates to processes and apparatuses for toluene methylation in an aromatics complex for producing paraxylene. More specifically, the present disclosure relates to processes and apparatuses for toluene methylation within an aromatics complex for producing paraxylene wherein an embodiment uses a riser reactor, another embodiment uses a pre-reactor producing dimethyl ether, and another embodiment uses partial regeneration of the catalyst.

### CROSS-REFERENCE TO RELATED APPLICATION

This application claims priority from Provisional Application No. 62/437,006 filed Dec. 20, 2016, the contents of which cited application are hereby incorporated by reference in its entirety.

### FIELD

This present disclosure relates to processes and apparatuses for toluene methylation in an aromatics complex for producing paraxylene. More specifically, the present disclosure relates to processes and apparatuses for toluene methylation within an aromatics complex for producing paraxylene wherein an embodiment uses a riser reactor, another embodiment uses a pre-reactor producing dimethyl ether, and another embodiment uses partial regeneration of the catalyst.

### BACKGROUND

The xylene isomers are produced in large volumes from petroleum as feedstocks for a variety of important industrial chemicals. The most important of the xylene isomers is paraxylene, the principal feedstock for polyester, which continues to enjoy a high growth rate from large base demand. Ortho-xylene is used to produce phthalic anhydride, which supplies high-volume but relatively mature markets. Meta-xylene is used in lesser but growing volumes for such products as plasticizers, azo dyes and wood preservers. Ethylbenzene generally is present in xylene mixtures and is occasionally recovered for styrene production, but is usually considered a less-desirable component of C₈ aromatics.

Among the aromatic hydrocarbons, the overall importance of xylenes rivals that of benzene as a feedstock for industrial chemicals. Xylenes and benzene are produced from petroleum by reforming naphtha but not in sufficient volume to meet demand, thus conversion of other hydrocarbons is necessary to increase the yield of xylenes and benzene. Often toluene is de-alkylated to produce benzene or selectively disproportionated or transalkylated to yield benzene and C₈ aromatics from which the individual xylene isomers are recovered.

An aromatics complex flow scheme has been disclosed by Meyers in the HANDBOOK OF PETROLEUM REFINING PROCESSES, 2d. Edition in 1997 by McGraw-Hill, and is incorporated herein by reference.

Traditional aromatics complexes send toluene to a transalkylation zone to generate desirable xylene isomers via transalkylation of the toluene with A₉₊ components. A₉₊ components are present in both the reformate bottoms and the transalkylation effluent.

Paraxylene is most often produced from a feedstock which has a methyl to phenyl ratio of less than 2. As a result, the paraxylene production is limited by the available methyl groups in the feed. In addition, paraxylene production also typically produces benzene as a byproduct. Since paraxylene is more valuable than benzene and the other byproducts produced in an aromatics complex, there is a desire to maximize the paraxylene production from a given amount of feed. There are also cases where a paraxylene producer would prefer to avoid the production of benzene as a byproduct or paraxylene production. However, there are also cases where a paraxylene producer would prefer to limit the production of benzene as a byproduct of paraxylene production by making adjustments.

### SUMMARY

The present subject matter relates to processes and apparatuses for toluene methylation in an aromatics complex for producing paraxylene. More specifically, the present disclosure relates to processes and apparatuses for toluene methylation in an aromatics complex for producing paraxylene. More specifically, the present disclosure relates to processes and apparatuses for toluene methylation within an aromatics complex for producing paraxylene wherein an embodiment uses a riser reactor, another embodiment uses a pre-reactor producing dimethyl ether, and another embodiment uses partial regeneration of the catalyst.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### Definitions

As used herein, the term “stream”, “feed”, “product”, “part” or “portion” can include various hydrocarbon molecules, such as straight-chain, branched, or cyclic alkanes, alkenes, alkadienes, and alkynes, and optionally other substances, such as gases, e.g., hydrogen, or impurities, such as heavy metals, and sulfur and nitrogen compounds. Each of the above may also include aromatic and non-aromatic hydrocarbons.

Hydrocarbon molecules may be abbreviated C₁, C₂, C₃, Cn where “n” represents the number of carbon atoms in the one or more hydrocarbon molecules or the abbreviation may be used as an adjective for, e.g., non-aromatics or compounds. Similarly, aromatic compounds may be abbreviated A₆, A₇, A₈, An where “n” represents the number of carbon atoms in the one or more aromatic molecules. Furthermore, a superscript “+” or “−” may be used with an abbreviated one or more hydrocarbons notation, e.g., C₃₊ or C₃₋, which is inclusive of the abbreviated one or more hydrocarbons. As an example, the abbreviation “C₃₊” means one or more hydrocarbon molecules of three or more carbon atoms.

As used herein, the term “zone” can refer to an area including one or more equipment items and/or one or more sub-zones. Equipment items can include, but are not limited to, one or more reactors or reactor vessels, separation vessels, distillation towers, heaters, exchangers, pipes, pumps, compressors, and controllers. Additionally, an equipment item, such as a reactor, dryer, or vessel, can further include one or more zones or sub-zones.

As used herein, the term “rich” can mean an amount of at least generally 50%, and preferably 70%, by mole, of a compound or class of compounds in a stream.

### BRIEF DESCRIPTION OF THE DRAWINGS

FIG. 1 illustrates a toluene methylation riser reactor having a mixing chamber.

FIG. 2 illustrates a toluene methylation dimethyl ether pre-reactor having staged injection.

FIG. 3 illustrates a toluene methylation partial regeneration scheme.

FIG. 4 illustrates partial regeneration of a spent catalyst to retain up to 2 wt % coke on catalyst back to the riser.

FIG. 5 illustrates partial regeneration of a spent catalyst to retain up to 6 wt % coke on catalyst back to the riser.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present disclosure. Also, common but well-understood elements that are useful or necessary in a commercially feasible embodiment are often not depicted in order to facilitate a less obstructed view of these various embodiments of the present disclosure.

### DETAILED DESCRIPTION

The following description is not to be taken in a limiting sense, but is made merely for the purpose of describing the general principles of exemplary aspects. The scope of the present disclosure should be determined with reference to the claims.

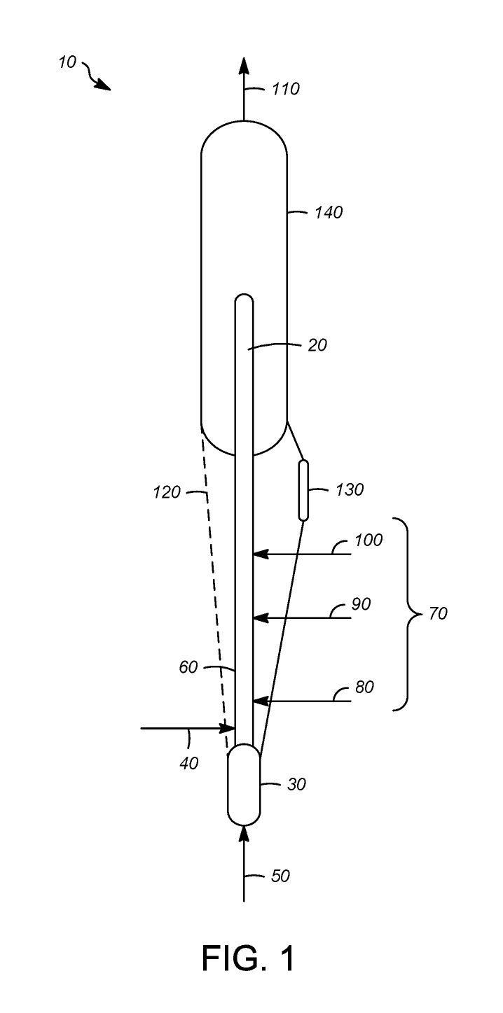

FIG. 1 illustrates a toluene methylation system 10 having a riser reactor 20 and a mixing chamber 30. FIG. 1 illustrates a process for alkylating an aromatic hydrocarbon reactant with an alkylating reagent comprising methanol to produce an alkylated aromatic product, comprising introducing the aromatic hydrocarbon feed 40 above the mixing chamber 30 comprising water 50. The aromatic hydrocarbon may also be injected directly into the riser 20. Additional streams are introduced into the riser reactor 20 which include methanol, toluene, and water. In the example illustrated in FIG. 1, there are a plurality of injection points 70 into the riser portion 60 of the riser reactor 20. In one embodiment there may be three injection points. The first injection point 80 may comprise a mixture of toluene, methanol, and water. The second injection point 90 and the third injection point 100 may comprise only methanol and water. The aromatic hydrocarbon may include a residence time of about 0.5 seconds to about 6 seconds, for producing the alkylated aromatic product. The product stream 110 may include an alkylated aromatic product includes xylene. Some of the coked catalyst from the reactor 140 may be recirculated to the mixing chamber 30 via line 120. Alternatively, a fraction of coked catalyst is cooled in a cooler 130 to remove heat of reaction and returned to the mixing chamber via the riser 60. The riser reactor 20 comprises a temperature of about 500° C. to about 700° C. The riser reactor 20 comprises an operating bed density of about 0.05 kg/m3 to 0.29 kg/m3. The weight hourly space velocity of the riser reactor 20 is about 4 hr−1 to about 20 hr−1. The weight hourly space velocity of the riser reactor is about 10 hr−1.

In an embodiment, the system 10 further includes passing the alkylate aromatic product 110 to a light olefins column to produce a light olefins product stream. Then the light olefins product stream may be passed to a toluene column to produce a toluene column product stream comprising paraxylene. In another embodiment the process 10 may include passing the light olefins product stream to a toluene column to produce a toluene column product stream comprising unreacted toluene and to recycle unreacted toluene to the reactor. The catalyst may include a MFI zeolite with silica-to-alumina ratio higher than 20, preferentially higher than 100, a silica or an alumina binder, or combined aluminosilicate binder; and a clay binder. In one embodiment, phosphorus is added to the catalyst. The MFI zeolite content in the catalyst is in the range of about 25 wt % to about 65 wt %. The catalyst may be in a powder format with an average particle size of about 70 microns to about 80 microns.

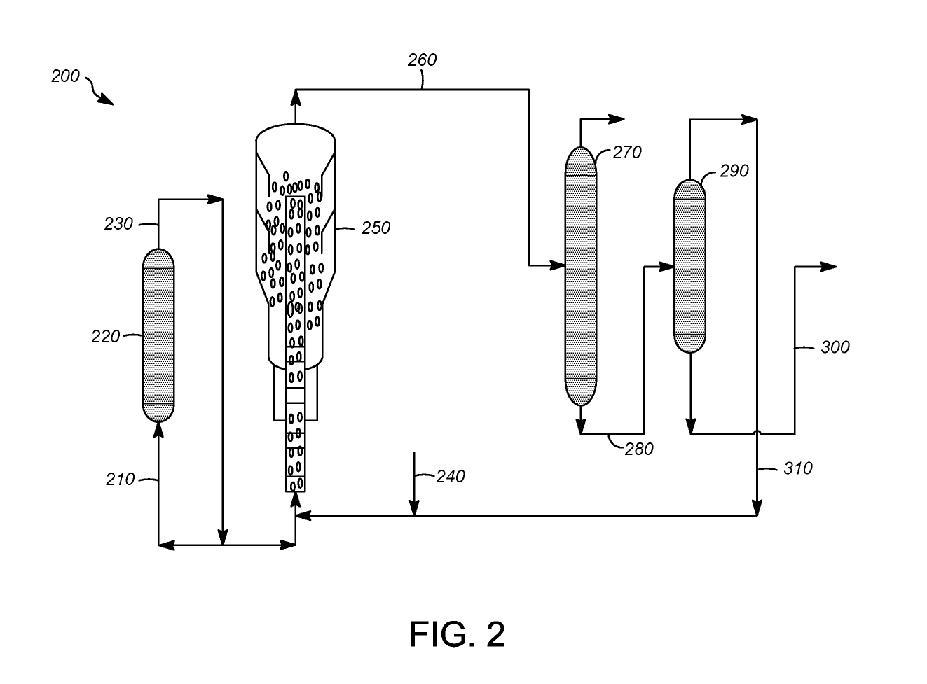

FIG. 2 illustrates a process 200 for alkylating an aromatic hydrocarbon reactant with an alkylating reagent comprising methanol to produce an alkylated aromatic product. The process 200 in FIG. 2 includes passing methanol 210 into a pre-reactor 220 to produce dimethyl ether and water 230, passing dimethyl ether and water 230 and toluene 240 to a riser reactor system 250 for producing the alkylated aromatic product 260. The a residence time in the reactor 250 may be about 0.5 seconds to about 6 seconds. The aromatic hydrocarbon reactant includes toluene, the alkylating reagent includes methanol, and the alkylated aromatic product 260 includes xylene.

The pre-reactor operates at about 400° C. to about 500° C. The pre-reactor comprises an operating bed density of about 0.30 kg/m3 to about 0.80 kg/m3. In some embodiments, the residence time in the riser reactor is 4 seconds. The weight hourly space velocity of the riser reactor is about 4 to about 20. The weight hourly space velocity of the riser reactor is about 10 hr−1. The riser reactor system comprises a temperature of about 500° C. to about 700° C. The riser reactor system comprises an operating bed density of about 0.05 kg/m3 to 0.29 kg/m3.

The pre-reactor may include a plurality of injection zones. The riser reactor may also include a plurality of injection zones, as illustrated in the example in FIG. 1. It is contemplated that the riser reactor comprises about 1 to about 4 injection points. It is also contemplated that the riser reactor may comprise 2 injection points.

In an embodiment, the system 200 further includes passing the alkylate aromatic product 260 to a light olefins column 270 to produce a light olefins product stream 280. Then the light olefins product stream 280 may be passed to a toluene column 290 to produce a toluene column product stream 300 comprising paraxylene. In another embodiment the process 200 may include passing the light olefins product stream 270 to a toluene column 290 to produce a toluene column product stream comprising unreacted toluene 310 and to recycle unreacted toluene 310 to the reactor 250. The catalyst may include a MFI zeolite with silica-to-alumina ratio higher than 20, preferentially higher than 100; a silica or an alumina binder, or combined aluminosilicate binder; and a clay binder. In one embodiment, phosphorus is added to the catalyst. The MFI zeolite content in the catalyst is in the range of about 25 wt % to about 65 wt %. The catalyst may be in a powder format with an average particle size of about 70 microns to about 80 microns.

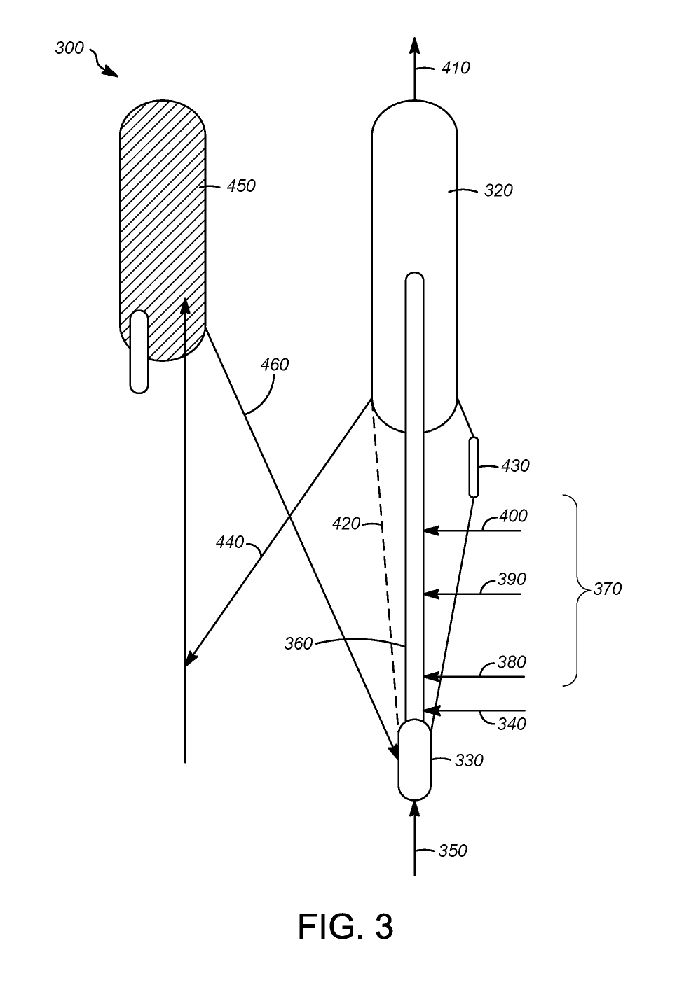

FIG. 3 illustrates a toluene methylation system 300 having a riser reactor 320, a mixing chamber 330, and a regenerator 450. More specifically, FIG. 3 illustrates a process for alkylating an aromatic hydrocarbon reactant with an alkylating reagent comprising methanol to produce an alkylated aromatic product, comprising introducing the aromatic hydrocarbon feed 340 above a mixing chamber 330 comprising water 350, and passing a portion of the coked catalyst 440 to the regenerator 450. Additional streams are introduced into the riser reactor system 320 which include methanol, toluene, and water. In the example illustrated in FIG. 3, there are a plurality of injection points 370 into the riser portion 360 of the riser reactor 320. In one embodiment there may be three injection points. The first injection point 380 may comprise a mixture of toluene, methanol, and water. The second injection point 390 and the third injection point 100 may comprise only methanol and water. The aromatic hydrocarbon may include a residence time of about 0.5 seconds to about 6 seconds, for producing the alkylated aromatic product. The product stream 410 may include an alkylated aromatic product includes xylene. Some of the coked catalyst from the reactor 320 may be recirculated to the mixing chamber 330 via line 420. Alternatively, a fraction of coked catalyst is cooled in a cooler 430 to remove heat of reaction and returned to the mixing chamber via the riser 360. The riser reactor 320 comprises a temperature of about 500° C. to about 700° C. The riser reactor 320 comprises an operating bed density of about 0.05 kg/m3 to 0.29 kg/m3. The weight hourly space velocity of the riser reactor 320 is about 4 hr−1 to about 20 hr−1. The weight hourly space velocity of the riser reactor is about 10 hr−1.

In an embodiment, the regenerator 450 produces a product stream of catalyst 460 wherein about 0.1% to about 15% of coke is left on the catalyst and the partially regenerated catalyst 460 is returned to the riser reactor 320. In a preferred embodiment, the regenerator 450 produces a product stream of catalyst 460 wherein about 2% to about 4% of coke is left on the catalyst and the partially regenerated catalyst 460 is returned to the riser reactor 320. In one embodiment, the regenerator 450 is a bubbling bed regenerator. In another embodiment, the regenerator 450 is a swing bed regenerator. In another embodiment, the regenerator 450 is a fixed bed regenerator. The oxygen concentration may be about 0.5% to about 21.0%.

In an embodiment, the system 300 further includes passing the alkylate aromatic product 410 to a light olefins column to produce a light olefins product stream. Then the light olefins product stream may be passed to a toluene column to produce a toluene column product stream comprising paraxylene. In another embodiment the process 300 may include passing the light olefins product stream to a toluene column to produce a toluene column product stream comprising unreacted toluene and to recycle unreacted toluene to the reactor. The catalyst may include a WI zeolite with silica-to-alumina ratio higher than 20, preferentially higher than 100; a silica or an alumina binder, or combined aluminosilicate binder; and a clay. In one embodiment, phosphorus is added to the catalyst. The MFI zeolite content in the catalyst is in the range of about 25 wt % to about 65 wt %. The catalyst may be in a powder format with an average particle size of about 70 microns to about 80 microns.

### Examples

The following examples are intended to further illustrate the subject embodiments.

These illustrations of different embodiments are not meant to limit the claims to the particular details of these examples.

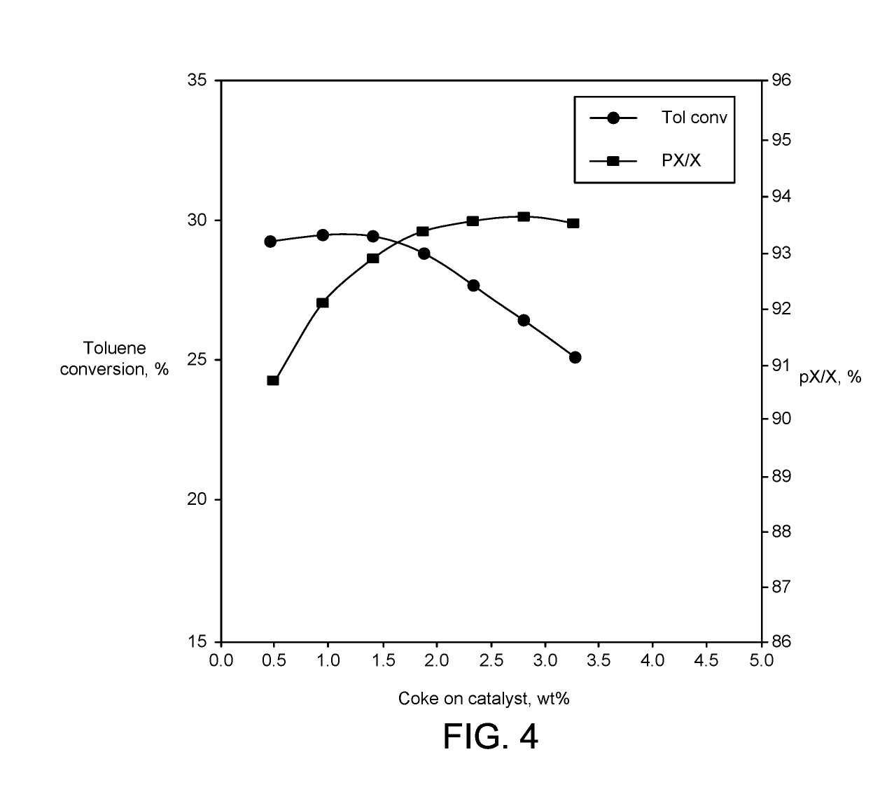

FIG. 4 illustrates that partial regeneration of a spent catalyst to retain up to 2 wt % coke on catalyst back to the riser would improve 2-3% PX/X selectivity. Optimal partial regeneration level leaves a residual level of coke that suppresses back-isomerization that would reduce PX concentration from well above equilibrium towards equilibrium. The catalyst comprises 40 wt % MFI zeolite with silica-to-alumina ratio of 500 and was steamed under 1050° C. for 90 minutes.

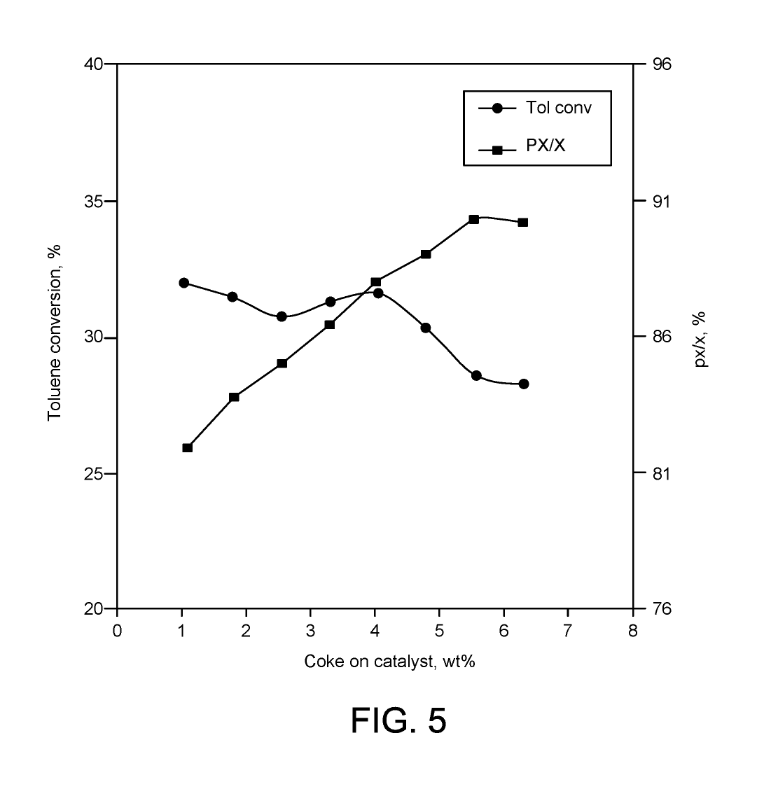

FIG. 5 illustrates that partial regeneration of a spent catalyst to retain up to 6 wt % coke on catalyst back to the riser would improve 3-5% PX/X selectivity. Additional residual coke levels above 2% and up to 6% allows PX/X to continue to increase without a significant and adverse effect on catalyst activity allowing PX/X to be maximized will still maintaining an acceptable toluene conversion. The catalyst comprises 40 wt % MFI zeolite with silica-to-alumina ratio of 500 and was steamed under 1050° C. for 45 minutes.

It should be noted that various changes and modifications to the presently preferred embodiments described herein will be apparent to those skilled in the art. Such changes and modifications may be made without departing from the spirit and scope of the present subject matter and without diminishing its attendant advantages.

### Specific Embodiments

While the following is described in conjunction with specific embodiments, it will be understood that this description is intended to illustrate and not limit the scope of the preceding description and the appended claims.

A first embodiment of the invention is a process for alkylating an aromatic hydrocarbon reactant with an alkylating reagent comprising methanol to produce an alkylated aromatic product, comprising passing methanol into a pre-reactor to produce dimethyl ether and water; passing dimethyl ether, water, and toluene to a into a riser reactor system, having a residence time of about 0.5 seconds to about 6 seconds, for producing the alkylated aromatic product; wherein the riser reactor system comprises an operating bed density of about 0.05 kg/m3 to 0.29 kg/m3; and recovering the alkylate aromatic product, produced by reaction of the aromatic reactant and the alkylating reagent, from the reactor system. An embodiment of the invention is one, any or all of prior embodiments in this paragraph up through the first embodiment in this paragraph, wherein the aromatic hydrocarbon reactant includes toluene, the alkylating reagent includes methanol and DME, and the alkylated aromatic product includes xylene. An embodiment of the invention is one, any or all of prior embodiments in this paragraph up through the first embodiment in this paragraph, wherein the pre-reactor operates at about 400° C. to about 500° C. An embodiment of the invention is one, any or all of prior embodiments in this paragraph up through the first embodiment in this paragraph, wherein the pre-reactor comprises an operating bed density of about 0.30 kg/m3 to 0.80 kg/m3. An embodiment of the invention is one, any or all of prior embodiments in this paragraph up through the first embodiment in this paragraph, wherein the residence time in the riser reactor is 4 seconds. An embodiment of the invention is one, any or all of prior embodiments in this paragraph up through the first embodiment in this paragraph, wherein the weight hourly space velocity of the riser reactor is about 4 to about 20. An embodiment of the invention is one, any or all of prior embodiments in this paragraph up through the first embodiment in this paragraph, wherein the weight hourly space velocity of the riser reactor is about 10 hr−1. An embodiment of the invention is one, any or all of prior embodiments in this paragraph up through the first embodiment in this paragraph, wherein the riser reactor system comprises a temperature of about 500° C. to about 700° C. An embodiment of the invention is one, any or all of prior embodiments in this paragraph up through the first embodiment in this paragraph, wherein the pre-reactor comprises a plurality of injection zones. An embodiment of the invention is one, any or all of prior embodiments in this paragraph up through the first embodiment in this paragraph, wherein the riser reactor comprises a plurality of injection zones. An embodiment of the invention is one, any or all of prior embodiments in this paragraph up through the first embodiment in this paragraph, wherein the riser reactor comprises about 1 to about 4 injection points. An embodiment of the invention is one, any or all of prior embodiments in this paragraph up through the first embodiment in this paragraph, wherein the riser reactor comprises 2 injection points. An embodiment of the invention is one, any or all of prior embodiments in this paragraph up through the first embodiment in this paragraph, further comprising passing the alkylate aromatic product to a light olefins column to produce a light olefins product stream. An embodiment of the invention is one, any or all of prior embodiments in this paragraph up through the first embodiment in this paragraph, further comprising passing the light olefins product stream to a toluene column to produce a toluene column product stream comprising paraxylene. An embodiment of the invention is one, any or all of prior embodiments in this paragraph up through the first embodiment in this paragraph, further comprising passing the light olefins product stream to a toluene column to produce a toluene column product stream comprising unreacted toluene and to recycle unreacted toluene to the reactor.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for alkylating an aromatic hydrocarbon reactant with an alkylating reagent comprising methanol to produce an alkylated aromatic product, comprising:
passing methanol into a pre-reactor to produce dimethyl ether and water;
passing dimethyl ether, water, and toluene to a into a riser reactor system, having a residence time of about 0.5 seconds to about 6 seconds, for producing the alkylated aromatic product;
wherein the riser reactor system comprises an operating bed density of about 0.05 kg/m³ to 0.29 kg/m³; and
recovering the alkylate aromatic product, produced by reaction of the aromatic reactant and the alkylating reagent, from the reactor system.

2. The process of claim 1, wherein the aromatic hydrocarbon reactant includes toluene, the alkylating reagent includes methanol and DME, and the alkylated aromatic product includes xylene.

3. The process of claim 1, wherein the pre-reactor operates at about 400° C. to about 500° C.

4. The process of claim 1, wherein the pre-reactor comprises an operating bed density of about 0.30 kg/m³ to 0.80 kg/m³.

5. The process of claim 1, wherein the residence time in the riser reactor is 4 seconds.

6. The process of claim 1, wherein the weight hourly space velocity of the riser reactor is about 4 to about 20.

7. The process of claim 1, wherein the weight hourly space velocity of the riser reactor is about 10 hr⁻¹.

8. The process of claim 1, wherein the riser reactor system comprises a temperature of about 500° C. to about 700° C.

9. The process of claim 1, wherein the pre-reactor comprises a plurality of injection zones.

10. The process of claim 1, wherein the riser reactor comprises a plurality of injection zones.

11. The process of claim 1, wherein the riser reactor comprises about one to about four injection points.

12. The process of claim 1, wherein the riser reactor comprises two injection points.

13. The process of claim 1, further comprising passing the alkylate aromatic product to a light olefins column to produce a light olefins product stream.

14. The process of claim 1, further comprising passing the light olefins product stream to a toluene column to produce a toluene column product stream comprising paraxylene.

15. The process of claim 1, further comprising passing the light olefins product stream to a toluene column to produce a toluene column product stream comprising unreacted toluene and to recycle unreacted toluene to the reactor.
